(19) United States
(12) Patent Application Publication
(10) Pub. No.: US 2012/0078338 A1
(43) Pub. Date: Mar. 29, 2012

(54) Shoe Electrode

(76) Inventor: David Sheraton, San Clemente, CA (US)

(21) Appl. No.: 13/237,267

(22) Filed: Sep. 20, 2011

Related U.S. Application Data
(60) Provisional application No. 61/387,384, filed on Sep. 28, 2010.

(51) Int. Cl. A61N 1/14; A61N 1/04

(52) U.S. Cl. 607/144

(57) ABSTRACT

A shoe electrode comprising an electrode, a skin adhesive for attaching the electrode to the skin, an electrically conductive heel pad, and a conductive pathway between the electrode and the heel pad. A removable adhesive is used to attach the heel pad to the heel of a shoe and in one embodiment, the electrode, heel pad and conductive pathway are formed as a single piece of conductive vinyl film.

### CROSS REFERENCE TO RELATED APPLICATIONS

This application claims the benefit of and priority to U.S. Provisional Application Ser. No. 61/387,384, filed Sep. 28, 2010, entitled, “SHOE ELECTRODE,” the content of which is incorporated by reference herein in its entirety.

### FIELD

The subject disclosure relates generally to medical devices and more particularly to a shoe electrode configured to conductively couple the human body to earth to achieve health benefits such as speeding recovery from acute trauma and/or preventing or reducing chronic inflammation.

### SUMMARY

According to an illustrative embodiment, apparatus for conductively coupling a human body to earth is provided comprising an electrode, a skin adhesive for attaching the electrode to the skin, an electrically conductive heel pad, and a conductive pathway between the electrode and the heel pad. A removable adhesive may be used to attach the heel pad to the heel of a shoe. In one embodiment, the electrode, heel pad and conductive pathway may be formed as a single piece of conductive vinyl film, and the skin adhesive may be a conductive hydrogel skin adhesive.

### DESCRIPTION OF THE DRAWINGS

FIG. 1 is a side plan view of a first illustrative shoe electrode embodiment; and

FIG. 2 is a side plan view of a second illustrative shoe electrode embodiment.

### DETAILED DESCRIPTION

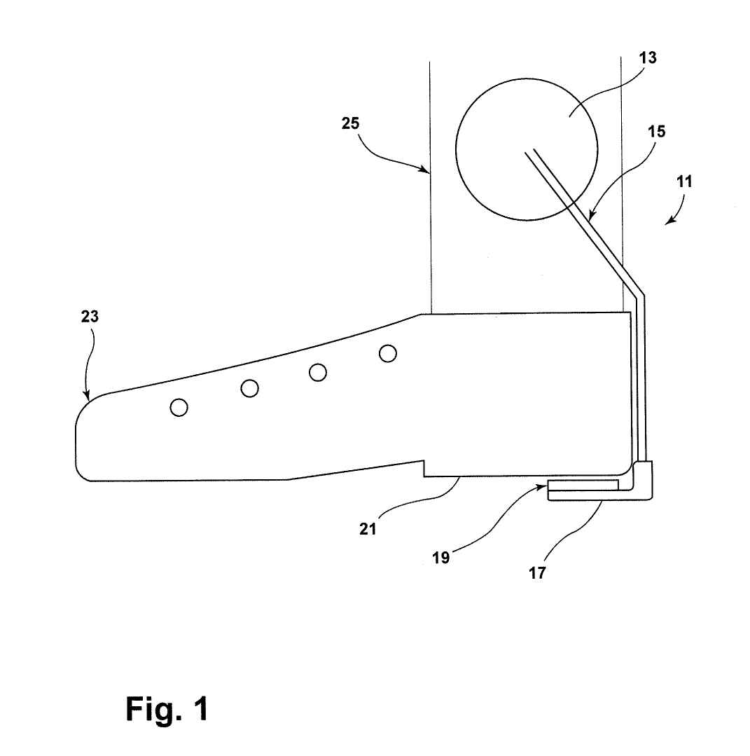

An illustrative shoe electrode embodiment 11 is shown in FIG. 1. This embodiment 11 includes a hydrogel electrode 13, an electrically conductive pathway 15, a heel pad 17 formed of electrically conductive material, and a removable adhesive layer 19. In this embodiment, the hydrogel electrode 13 is attached just above the ankle

The removable adhesive layer 19 attaches the heel pad 17 to the heel 21 of a shoe 23, while the hydrogel electrode 13 attaches to the skin 25 of a human wearing the shoe 23. A conductive path is thus created from the earth through the conductive heel material 17 and then through the conductive pathway 15 and electrode 13 to the body of the wearer of the shoe 23.

In operation, when the human body is conductively coupled to the earth by the electrode device 11, the body equalizes with and maintains the natural electrical potential of the earth. In this state, the earth's mobile free electrons are available throughout the human body to readily reduce excess free radicals and thereby prevent oxidation of healthy tissue. Restoring the earth's natural surface charge of free electrons to the body assists in speeding recovery from acute trauma and preventing or reducing chronic inflammation. Thus, in one embodiment, electrode apparatus according to the illustrative embodiments is attached to the human wearer for a period of time selected to achieve a beneficial or therapeutic response.

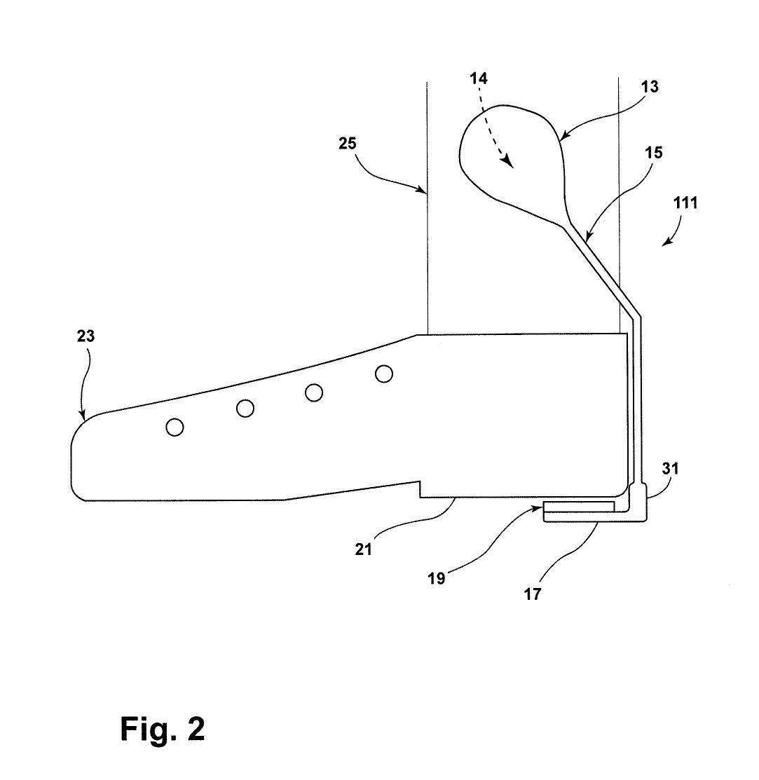

In another illustrative embodiment 111, shown in FIG. 2 a single piece of conductive metal printed film 31 runs the length of the device and provides the heel pad 17, pathway 15, and the conductive portion or backing of the electrode 13. Conductive hydrogel skin adhesive 14 is applied at the electrode end of the conductive film 31 to attach it to the skin 25, and a removable heel adhesive 19 is used at the other end to attach the conductive film 31 to the shoe heel 21. In one embodiment, the single piece of metal printed film 31 may be die cut to a functional shape.

In the embodiment of the device 111 of FIG. 2, the conductive skin adhesive 14 may be between 10 and 60 mils thick and may be, for example, Comfort Gel A as made by R&D Medical Products, Inc., Lake Forest, Calif. The conductive metal printed film 31 may be, for example, a silver printed carbon loaded vinyl film, 0.5 to 6 mils thick such; as “Inspire 4201” made by Exopack Advanced Coatings. The removable adhesive 19 may be a 1 to 10 mils thick adhesive layer such as, for example, 3M 1524 made by 3M Corporation, Minneapolis, Minn.

In some embodiments an electrical connector may be inserted into the conductive pathway , e.g. 31, so that an upper portion of the pathway can be disconnected from a lower portion. Such a connector may be, for example, a thin wire pin socket such as would fit through a stocking or a flex circuit ZIF connector. Such a feature provides for replacement of the body or the heel portion of electrode if one lasts longer than the other. In one such embodiment, the heel electrode could be relatively permanent and the body electrode relatively disposable. The conductive pathway length may also be variable in the event that there is a need to treat a part of the body far above the ankle

From the foregoing, those skilled in the art will appreciate that various adaptations and modifications of the just described preferred embodiment can be configured without departing from the scope and spirit of the invention. Therefore, it is to be understood that, within the scope of the appended claims, the invention may be practiced other than as specifically described herein.

What is claimed is:

1. A medical device for conductively coupling a human body to earth comprising:
an electrode attached to the skin;
an electrically conductive heel pad; and
a conductive pathway between said electrode and said heel pad.

2. The apparatus of claim 1 wherein said electrode, heel pad and conductive pathway are formed as a single piece of conductive film.

3. The apparatus of claim 2 wherein said conductive film comprises a metal printed film.

4. The apparatus of claim 1 further comprising a skin adhesive for attaching the electrode to the skin.

5. The apparatus of claim 4 wherein said skin adhesive comprises conductive hydrogel skin adhesive.

6. The apparatus of claim 1 further comprising a removable heel adhesive.

7. The apparatus of claim 5 further comprising a removable heel adhesive.

8. A method comprising:
providing apparatus for establishing a conductive path which conductively couples a human body to earth, the apparatus comprising an electrode; an electrically conductive heel pad;
and a conductive pathway interconnecting said electrode and said heel pad;
applying a skin adhesive to attach said electrode to the skin; and
applying a removable heel adhesive to attach said heel pad to a shoe;
wherein said conductive path is maintained for a period of time selected to achieve a beneficial therapeutic effect on the human body.

9. The apparatus of claim 7 wherein said electrode, heel pad and conductive pathway are formed as a single piece of conductive film.

10. The apparatus of claim 8 wherein said conductive film comprises a metal printed film.

11. The apparatus of claim 9 wherein said skin adhesive comprises conductive hydrogel skin adhesive.
